(19) United States
(12) Patent Application Publication
(10) Pub. No.: US 2025/0387040 A1
(43) Pub. Date: Dec. 25, 2025

(54) ASCERTAINING THE POSITION OF A PATIENT IN A MAGNETIC RESONANCE APPARATUS

(71) Applicant: Siemens Healthineers AG, Forchheim (DE)

(72) Inventors: David Grodzki, Erlangen (DE); Stephan Biber, Erlangen (DE)

(21) Appl. No.: 19/243,200

(22) Filed: Jun. 19, 2025

(30) Foreign Application Priority Data
Jun. 21, 2024 (DE) 10 2024 205 802.5

(51) Int. Cl. A61B 5/055; A61B 5/11; G01R 33/385

(52) U.S. Cl. CPC A61B 5/055; A61B 5/1128; G01R 33/385

(57) ABSTRACT

Systems and methods for ascertaining an item of position information about a position of a patient positioned in a magnetic resonance apparatus are provided. The magnetic resonance apparatus includes at least one transmit antenna, wherein each of the at least one transmit antennas is provided to generate RF transmit pulses for generating magnetic resonance signals in the body of the patient. Furthermore, the magnetic resonance apparatus includes at least one receive antenna that is configured to receive a response signal of the RF transmit pulse, for example a magnetic resonance signal triggered by the RF transmit pulse. An RF transmit pulse is generated with the at least one transmit antenna of the magnetic resonance apparatus, which pulse includes a frequency band which is outside of an examination frequency band of the magnetic resonance apparatus. The item of position information is ascertained using the response signal of the RF transmit pulse.

### CROSS REFERENCE TO RELATED APPLICATIONS

This application claims the benefit of DE 10 2024 205 802.5 filed on Jun. 21, 2024, which is hereby incorporated by reference in its entirety.

### FIELD

Embodiments relate to a method for ascertaining an item of position information about a position of a patient positioned in a magnetic resonance apparatus, to a magnetic resonance apparatus and to a computer program product.

### BACKGROUND

In medical engineering, imaging by magnetic resonance (MR), also called magnetic resonance imaging (MRI), is characterized by high soft tissue contrasts. A magnetic resonance examination, for example a magnetic resonance measurement, is carried out by a magnetic resonance apparatus, that includes a magnet unit. The magnet unit y includes a main magnet for generating a main magnetic field and a gradient coil unit for generating a gradient magnetic field in an examination region of the magnetic resonance apparatus. The examination region is customarily situated in a magnet bore of the magnet unit, in which a patient is positioned during the magnetic resonance measurement. For generating imaging and/or spectroscopic magnetic resonance signals during a magnetic resonance measurement, radio-frequency (RF) transmit pulses are irradiated by one or more transmit antenna(s) in accordance with a measurement protocol, for example a magnetic resonance sequence, into the examination region, for example into the patient. The RF transmit pulses, that are generated for generating the imaging and/or spectroscopic magnetic resonance signals, customarily have an examination frequency band.

The irradiation of the RF transmit pulses generates RF fields with which nuclear spins in the patient are deflected from their rest position. A subsequent relaxation generates imaging and/or spectroscopic magnetic resonance signals that are received by one or more receive antenna(s) of the magnetic resonance apparatus and are used for reconstruction of magnetic resonance mappings or for spectroscopy.

The generated RF fields cause heating of the tissue of the patient, which is described by a specific absorption rate (SAR). Local intensity maxima may occur primarily in the immediate vicinity of the transmit antennas, which maxima may in the worst case cause burns to the patient. The transmit antennas may be part of a body coil permanently integrated in the magnetic resonance apparatus and that is situated immediately behind an inner wall of a magnet bore of the magnetic resonance apparatus. The transmit power is therefore limited by what is known as contact protection.

Since, according to the prior art, there is no information about the current position of the patient and their actual distances from the inner wall of the magnet bore, often very conservative safety measures are used.

### BRIEF SUMMARY AND DESCRIPTION

The scope of the embodiments is defined solely by the appended claims and is not affected to any degree by the statements within this summary. The present embodiments may obviate one or more of the drawbacks or limitations in the related art. Independent of the grammatical term usage, individuals with male, female or other gender identities are included within the term.

Embodiments ascertain the position of a patient in order to make, for example, improved SAR monitoring possible.

Accordingly, a method for ascertaining an item of position information about a position of a patient positioned in a main magnetic field of a magnetic resonance apparatus is provided. The magnetic resonance apparatus includes at least one transmit antenna, wherein each of the at least one transmit antennas is provided, for example configured, to generate RF transmit pulses for generating magnetic resonance signals in the body of the patient. Furthermore, the magnetic resonance apparatus includes at least one receive antenna that is configured to receive a response signal of the RF transmit pulse, for example a magnetic resonance signal triggered by the RF transmit pulse. The at least one transmit antenna of the magnetic resonance apparatus generates an RF transmit pulse that includes a frequency band that is outside of an examination frequency band of the magnetic resonance apparatus (hereinafter also called position RF transmit pulse). The item of position information is ascertained using the (possible) response signal of the RF transmit pulse.

The item of position information may be ascertained, for example, with an ascertainment unit. The ascertainment unit may, for example, be part of a system control unit of the magnetic resonance apparatus. The ascertainment unit may, for example, include one or more processor(s) and/or one or more memory module(s).

The ascertained position may be provided and/or saved. For example, the ascertained position may be displayed and/or processed further. For example, a control signal may be generated using the item of position information.

Hereinafter the RF transmit pulse will also be called a position RF transmit pulse in order to clearly differentiate it from any further RF transmit pulses that are not used for ascertaining the item of position information, but instead, for example, for generating imaging and/or spectroscopic magnetic resonance signals; such further RF transmit pulses customarily have the examination frequency band. such further RF transmit pulses are may also be generated with the same at least one transmit antenna with which the position RF transmit pulses are also generated.

The response signal may be a magnetic resonance signal (potentially) triggered by the position RF transmit pulse, for example a signal of a free induction decay (FID) signal. The response signal itself may not be suitable and/or provided for reconstructing magnetic resonance imaging therefrom and/or deriving an item of spectroscopic information therefrom. The response signal itself may not include any medical-diagnostic information.

The item of position information may include, for example, whether a response signal was received or not and/or derived therefrom. The item of position information may include, for example, a property of the response signal, such as the strength of the response signal, and/or be derived therefrom.

The magnetic resonance apparatus may include, for example, a tunnel-shaped and/or a cylindrical, magnet bore in which at least one part of the patient is positioned. The region of the magnet bore may be a patient-receiving region in which the at least one part of the patient is positioned. For example, the part of the patient from which imaging and/or spectroscopic magnetic resonance signals are to be captured with the magnetic resonance apparatus is positioned in the magnet bore. For example, at least one part of the patient is positioned in an isocenter of the magnetic resonance apparatus, for example of the main magnetic field. In the isocenter the main magnetic field, for example, the strength of the main magnetic field, may have a particularly high homogeneity. For example, in the isocenter the main magnetic field includes a homogeneity of less than 2 ppm.

The magnet bore may be delimited by an inner wall. The inner wall may be situated between a patient-receiving region in which at least one part of the patient is positioned, and the at least one receive antenna. For example, the at least one receive antenna is a constituent part of a body coil that is permanently integrated in a magnet unit of the magnetic resonance apparatus. The magnet unit may surround the patient-receiving region and/or delimit the magnet bore.

The examination frequency band of the magnetic resonance apparatus includes the frequencies with which, for generating imaging and/or spectroscopic magnetic resonance signals, nuclear spins precess around the direction of an outer magnetic field (hereinafter also called Larmor frequency, resonance frequency and/or precession frequency). Frequencies outside of the examination frequency band of the magnetic resonance apparatus may also be referred to as off-resonant. Outside of the examination frequency band are frequency ranges that are outside of the frequency range customarily used for imaging and/or spectroscopy.

The Larmor frequency is dependent on the nucleus type and the strength of the applied magnetic field, for example of the main magnetic field. At 1.0 tesla the Larmor frequency of protons is, for example, approx. 42 MHz, at 1.5 tesla approx. 63 MHz. The Larmor frequency may be calculated with equation (1): f=γ/(2π)B, where γ is the gyromagnetic ratio and B the strength of the magnetic field.

An appropriate Larmor frequency may therefore be associated with a magnetic field strength. With a homogeneous magnetic field strength, the associated Larmor frequency is therefore also homogeneous. Consequently the associated Larmor frequency also includes a high homogeneity in the isocenter.

Customarily, there is a localized dependence of the strength of the main magnetic field. As already explained, in the region of the isocenter the strength of the main magnetic field is customarily particularly homogeneous. Customarily, the strength of the main magnetic field is increasingly more inhomogeneous outwardly, for example in the direction of the inner wall of the magnet bore. There is thus a correlation between location and strength of the magnetic field, for example of the main magnetic field.

Advantageously, this correlation, i.e. the spatial distribution of the strength of the main magnetic field, is known. Advantageously, the strength of the main magnetic field is provided for ascertaining the item of position information. Advantageously, the item of position information is ascertained using the spatial distribution of the strength of the main magnetic field.

Advantageously, the selection of the frequency band of the position RF transmit pulse makes it possible to determine in which spatial region nuclear spins, for example protons, are excited by magnetic resonance. The response signal results from such an excitation. For example, only nuclear spins, that are situated in a specific spatial region, for example outside of the isocenter, are excited with the position RF transmit pulse. For example, the response signal is a magnetic resonance signal of protons (for example in the body of the patient) that, in this specific spatial region, are situated, for example, outside of the isocenter. If the patient moves a body part, for example in such a way that it is situated in the specific region, for example outside of the isocenter, nuclear spins may be excited in this body part by the position RF transmit pulse. Advantageously, an item of position information may then be ascertained that includes an item of information that this body part is situated in the specific region, for example outside of the isocenter.

Advantageously, the size of the spatial region in which nuclear spins, for example protons, are excited by magnetic resonance, may also be determined via the width of the frequency band of the position RF transmit pulse. The wider the frequency band of the RF transmit pulse is, the larger the spatial region monitored by the RF transmit pulse also customarily is with a constant magnetic field distribution, since equation (1) is satisfied for a plurality of frequencies and thereby a plurality of locations.

The frequency band of the position RF transmit pulse is outside of the examination frequency band of the magnetic resonance apparatus. A magnetic resonance excitation outside an imaging volume of the magnetic resonance apparatus takes place due to the position RF transmit pulse. By contrast, imaging and/or spectroscopic magnetic resonance signals are generated customarily with RF transmit pulses whose frequency components are in the examination frequency band. A magnetic resonance excitation takes place in the imaging volume of the magnetic resonance apparatus due to RF transmit pulses whose frequency components are in the examination frequency band.

Advantageously, the magnetic resonance apparatus includes a transmit system, for example including the at least one transmit antenna, that is configured to capture the position RF transmit pulse as well as RF transmit pulses for generating imaging and/or spectroscopic magnetic resonance signals. The transmit system includes a bandwidth within which lie the frequency band of the position RF transmit pulse and the examination frequency band of the magnetic resonance apparatus.

Advantageously, the magnetic resonance apparatus includes a receive system, for example including the at least one receive antennas, that is configured to capture the response signal of the position RF transmit pulses as well as imaging and/or spectroscopic magnetic resonance signals. The receive system includes a bandwidth within which lie the frequency band of the position RF transmit pulse and the examination frequency band of the magnetic resonance apparatus.

Advantageously, response signals, for example magnetic resonance signals, are acquired from strongly off-resonant regions and associated with the patient, from which it is possible to infer whether at least a part of the patient is situated in specific local regions or not. Advantageously, no additional hardware with respect to a conventional magnetic resonance apparatus is required for carrying out the method.

Advantageously, the frequency band of the RF transmit pulse includes a distance from the center frequency of the examination frequency band, with the distance being selected such that the response signal includes an item of position information about a spatial region of interest and/or that is to be monitored. For example, the frequency band of the RF transmit pulse is in a region of ±1,000-2,500 ppm relative to the center frequency of the examination frequency band. In this connection “ppm” means “parts per million” and/or “millionth”.

Advantageously, the distance is selected such that a spatial region that is relevant to the safety of the patient is covered thereby. Advantageously, the distance is selected such that a spatial region that constitutes a danger to the patient if the body of the patient is situated wholly or partially therein is covered thereby.

The frequency band of the RF transmit pulse includes a distance from the center frequency of the examination frequency band of the magnetic resonance apparatus of at least 100 ppm, for example at least 200 ppm, for example at least 500 ppm.

The item of position information is ascertained during a magnetic resonance measurement. For example, a measurement protocol, for example a magnetic resonance sequence, may be applied during the magnetic resonance measurement, according to which imaging and/or spectroscopic magnetic resonance signals are captured with the aid of RF transmit pulses in the examination frequency band and response signals are captured with the aid of position RF transmit pulses outside of the examination frequency band.

The imaging and/or spectroscopic magnetic resonance signals as well the response signals may be captured successively. Advantageously, the RF transmit pulses for generating the response signals are switched on between the other RF transmit pulses generated without interfering with the capture of the imaging and/or spectroscopic magnetic resonance signals.

Generation of the position RF transmit pulse and ascertainment of the item of position information may be carried out repeatedly during the magnetic resonance measurement of the patient. The more often response signals are generated and received, the more continuously the position of the patient may be monitored.

The frequency band of the RF transmit pulses may be varied with repeated generation of the position RF transmit pulse and ascertainment of the item of position information. Advantageously, it is thus possible to monitor different spatial regions. Thus, for example with a first position RF transmit pulse, that includes a first frequency band, it is possible to monitor a first region, and with a second position RF transmit pulse, that includes a second frequency band (different from the first frequency band), it is possible to monitor a second region (different from the first region).

Advantageously, a variation in the frequency band of the RF transmit pulse may improve the spatial resolution of the monitoring. For example, the frequency bands may be reduced, so the associated regions also become smaller. Conversely, it is possible to emit more RF transmit pulses in order to obtain the overall coverage of the monitoring.

The magnetic resonance apparatus may include a magnet bore within which at least one part of the patient is positioned, wherein the item of position information includes an item of distance information about the distance of the patient, for example any body part of the patient, from an inner wall of the magnet bore.

For example, the distance of the frequency band of the position RF transmit pulse from the center frequency of the examination frequency band is selected such that a possible response signal is generated in a region close to the inner wall of the magnet bore. If the patient moves, for example, a body part, for example an arm, into this region close to the inner wall of the magnet bore, the position RF transmit pulse may generate a response signal, for example a magnetic resonance signal, by interacting with nuclei, for example protons, in the body part. The response signal may be received by the magnetic resonance apparatus, and the item of position information may be ascertained using the response signal. Even if no response signal is received, an item of position information exists since it is possible to infer from the missing response signal that no tissue generating a response signal is situated in the spatial region of interest and/or that is to be monitored.

In this way it is possible to achieve contact protection of the inner wall of the magnet bore, for example delimitation of the transmit power. Instead of adopting a conservative safety assumption for the SAR monitoring, the actual position of the patient, for example their body parts, may be taken into account. Advantageously, more effective magnetic resonance protocols may be applied thereby.

One possible embodiment of the method provides that the magnetic resonance apparatus includes at least one gradient coil, wherein a magnetic field gradient is generated in the main magnetic field during the generation of the RF transmit pulse with the at least one gradient coil.

Advantageously, a slice-selective excitation of response signals by the position RF transmit pulse may be carried out by the generation of the magnetic field gradients. The item of position information may consequently be improved, for example the position of the patient may be ascertained more accurately.

For example, the magnet bore includes a center axis and/or a longitudinal axis (z-axis). This may run parallel to the surface of the magnet bore. (In the case of a circular cylindrical magnet bore, the center axis and/or longitudinal axis would run through the center of the circular base area of the cylinder.) The generated magnetic field gradient runs along the center axis and/or a longitudinal axis of the magnet bore. Advantageously, a slice may be selected in the z-direction with the aid of such a magnetic field gradient.

Advantageously, the item of position information may be improved by such a slice selection. For example, any excitation regions close to the inner wall of the magnet bore may thus be separated from those that have a greater distance from the inner wall of the magnet bore, but a different position along the z-axis.

One possible embodiment of the method provides that the at least one receive antenna includes a plurality of receive antennas whose positions relative to the magnetic resonance apparatus, for example to the magnet bore, are known. Each of the plurality of receive antennas is configured to receive a partial response signal of the response signal, with the item of position information being ascertained using the (possible) partial response signals and the positions of the receive antennas. Advantageously, the ascertained item of position information may consequently be improved.

The partial response signals may be received by the plurality of receive antennas. For example, each of the plurality of receive antennas may be associated with one receive channel. The magnetic resonance apparatus may include, for example, 8, 16 or 32 receive channels with one receive antenna respectively. The item of position information may be ascertained with the aid of a coil sensitivity profile of the plurality of receive antennas.

Advantageously, an item of information about the point of origin of the response signal may be derived from the known position of the respective receive antenna and the partial response signal received by the receive antenna. Advantageously, the partial response signals may be correlated with each other and/or compared to each other such that their point of origin may be inferred. All partial response signals of the response signal may have the same root cause, for example the same relaxation process of one or more nuclear spin(s) excited by a position RF pulse (for example of nuclei in the body of the patient, if it is situated in a corresponding region that is affected by magnetic resonance and generation of a response signal resulting therefrom).

If, for example, a first partial response signal, that was received by a first receive antenna, includes a larger amplitude than a second partial response signal, that was received by a second receive antenna, then it is possible to infer from this, for instance, that the point of origin of the response signal including the two partial response signals is located closer to the position of the first receive antenna.

The plurality of receive antennas may be constituent parts of one or more local coil(s). Local coils may be provided to be arranged directly on the body of the patient. The partial response signals are received by receive antennas in one or more local coil(s). The one or more local coil(s), for example the receive antennas, may be arranged directly on the body of the patient.

Furthermore, a magnetic resonance apparatus is provided that is configured to carry out a method as described above. The magnetic resonance apparatus may include, for example, at least one transmit antenna for generating RF transmit pulses and/or at least one receive antenna for receiving magnetic resonance signals. Furthermore, the magnetic resonance apparatus may include, for example, a magnet bore, inside of which at least one part of the patient may be positioned. Furthermore, the magnetic resonance apparatus may include, for example, a system control unit for controlling generation of RF transmit pulses, for evaluating received response signals of the RF transmit pulses, for example for ascertaining an item of position information using the response signals.

The advantages of the proposed magnetic resonance apparatus substantially correspond to the advantages of the method described above for ascertaining an item of position information about a position of a patient positioned in a main magnetic field of a magnetic resonance apparatus, that are stated above in detail. Features, advantages or alternative embodiments mentioned in this connection may likewise also be transferred to the other claimed subject matter, and vice versa.

Furthermore, a computer program product is provided that includes a program and may be loaded directly into a memory of a programmable system control unit of a magnetic resonance apparatus and includes program code, for example libraries and auxiliary functions, in order to carry out a proposed method when the computer program product is executed in the system control unit of the magnetic resonance apparatus. The computer program product may include software with a source code that still has to be compiled and linked or that only has to be interpreted, or executable software code that only has to be loaded into the system control unit for execution.

The method may advantageously be carried out quickly, in an identically repeatable manner and robustly by way of the computer program product. The computer program product may be configured such that it may execute the method steps by the system control unit. The system control unit has in each case the prerequisites such as an appropriate main memory, an appropriate graphics card or an appropriate logic unit, so the respective method steps may be efficiently carried out.

The computer program product is saved, for example, on a computer-readable medium or stored on a network or server from where it may be loaded into the processor of a local system control unit that is directly connected to the magnetic resonance apparatus or may be configured to be part of the magnetic resonance apparatus. Furthermore, items of control information of the computer program product may be saved on an electronically readable data carrier. The items of control information of the electronically readable data carrier may be configured in such a way that they carry out a proposed method when the data carrier is used in a system control unit of a magnetic resonance apparatus.

Examples of electronically readable data carriers are a DVD, a magnetic tape or a USB stick, on which electronically readable items of control information, for example software, are saved. When these items of control information are read from the data carrier and saved in a system control unit of the magnetic resonance apparatus, all proposed embodiments of the methods described above may be carried out.

Further advantages, features and details may be found in the embodiments described below, as well as on the basis of the drawings. Parts which correspond to one another are provided with the same reference characters in all the drawings.

### BRIEF DESCRIPTION OF THE FIGURES

FIG. 1 depicts a magnetic resonance apparatus in a schematic representation according to an embodiment.

FIG. 2 depicts a block diagram of a method for ascertaining an item of position information of a patient according to an embodiment.

FIGS. 3 and 6 depict representations of regions in a magnet bore, that may be monitored by the method according to an embodiment.

FIGS. 4 and 5 depict representations of an examination frequency band and two frequency bands of position RF transmit pulses arranged symmetrically around it according to an embodiment.

### DETAILED DESCRIPTION

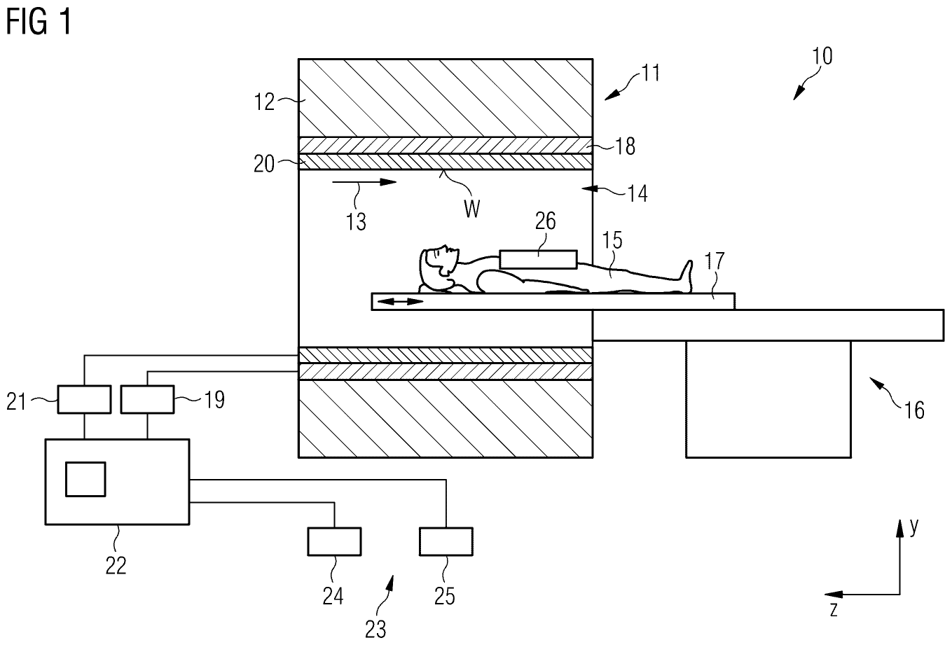

FIG. 1 schematically represents a magnetic resonance apparatus 10. The magnetic resonance apparatus 10 includes a magnet unit 11 that includes a main magnet 12 for generating a strong and, for example, temporally constant main magnetic field 13. In addition, the magnetic resonance apparatus 10 includes a magnet bore 14 for receiving a patient 15. The magnet bore 14 is configured in the form of a cylinder with a center axis in the z-direction and is surrounded by the magnet unit 11 in a circumferential direction. The magnet bore 14 is delimited by an inner wall W. The patient 15 may be pushed into the magnet bore 14 by a patient positioning apparatus 16 of the magnetic resonance apparatus 10. The patient positioning apparatus 16 includes for this purpose a patient table 17 configured to move inside the magnet bore 14.

The magnet unit 11 also includes a gradient coil unit 18 with at least one gradient coil for generating magnetic field gradients that are used for spatial encoding during imaging. The gradient coil unit 18 is controlled by a gradient control unit 19 of the magnetic resonance apparatus 10. The magnet unit 11 also includes a radio-frequency antenna unit 20 with at least one transmit antenna that in the present exemplary embodiment is configured as a body coil permanently integrated in the magnetic resonance apparatus 10. The radio-frequency antenna unit 20 is controlled by a radio-frequency antenna control unit 21 of the magnetic resonance apparatus 10 and irradiates radio-frequency magnetic resonance sequences, for example RF transmit pulses, into an examination space that is substantially formed by the region of the magnet bore 14 of the magnetic resonance apparatus 10. The main magnetic field 13 generated by the main magnet 12 establishes an excitation of atomic nuclei in that nuclear spins are deflected from their rest position. Magnetic resonance signals are generated due to relaxation of the excited atomic nuclei. The radio-frequency antenna unit 20 may basically be configured to receive the magnetic resonance signals. Here, a local coil 26 that is configured to receive the magnetic resonance signals is arranged on the patient 15.

The magnetic resonance apparatus 10 includes a system control unit 22 for controlling the main magnet 12, the gradient control unit 19 and for controlling the radio-frequency antenna control unit 21. The system control unit 22 centrally controls the magnetic resonance apparatus 10, such as carrying out a predetermined imaging gradient echo sequence. In addition, the system control unit 22 includes an evaluation unit (not shown) for evaluating the magnetic resonance signals that are captured during the magnetic resonance examination. Furthermore, the magnetic resonance apparatus 10 includes a user interface 23 that is connected to the system control unit 22. Items of control information, such as imaging parameters, as well as reconstructed magnetic resonance mappings, may be displayed on a display unit 24, for example on at least one monitor, of the user interface 23 for a medical operator. Furthermore, the user interface 23 includes an input unit 25 by which items of information and/or parameters may be input by the operator during a measuring process.

Irradiation of the RF transmit pulses by the radio-frequency antenna unit 20 may result in heating of the tissue of the patient 15, and this may be described by the specific absorption rate (SAR). Local intensity maxima and thus, in the worst cases, burns to the patient may occur primarily in the immediate vicinity of the radio-frequency antenna unit 20. According to the prior art, the transmit power of the radio-frequency antenna unit 20 is therefore limited by what is known as contact protection that is based on conservative assumptions of the actual position of the patient in the magnet bore. To be able to dispense with these assumptions it would be desirable to obtain detailed items of information about an actual position of the patient 15.

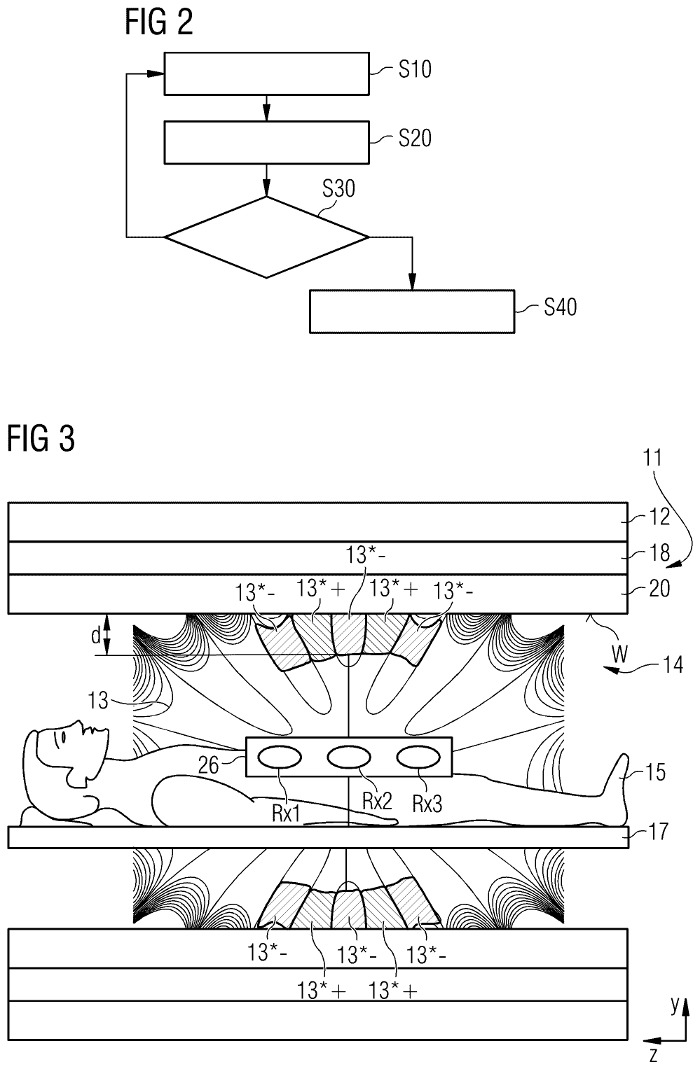

FIG. 2 represents a method for ascertaining an item of position information about a position of the patient 15 positioned in the main magnetic field 13 of the magnetic resonance apparatus 10. This method may be carried out during a magnetic resonance measurement of the patient 15. In S10, an RF transmit pulse is generated with the at least one transmit antenna of the radio-frequency antenna unit 20, with the RF transmit pulse having a frequency band that is located outside of an examination frequency band of the magnetic resonance apparatus. In S20, a response signal of the RF transmit pulse, for example an FID signal, is received by the at least one receive antenna of the local coil 26. In S30, the item of position information is ascertained using the response signal of the transmit pulse. If the item of position information is uncritical, a further item of position information is ascertained by repeating S10, S20 and S30. If the item of position information is critical, a suitable measure is carried out in S40. An item of position information may be critical, for example, if a minimum distance of the patient 15 from the inner wall W of the magnet bore 14 is not observed. Such a measure may be, for example, an interruption to the measurement or an adjustment of the magnetic resonance measurement, for example an adjustment of parameters of the magnetic resonance sequence to be applied.

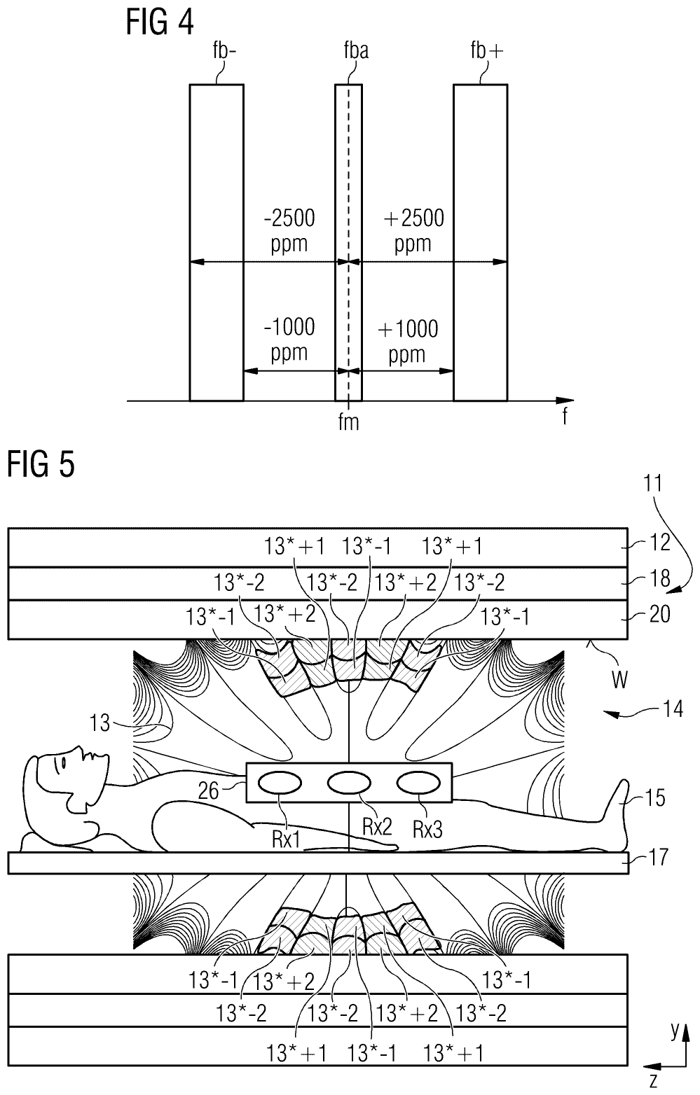

The method is explained in more detail below using FIGS. 3 to 6. FIG. 3 represents in more detail the region of the magnet bore 14 with the main magnetic field 13 situated therein. The represented lines show a location at which there is an identical strength of the main magnetic field 13. The closer together the lines are, the greater the inhomogeneity of the main magnetic field 13 is. Accordingly, in the center of the magnet bore the main magnetic field 13 includes a particularly high homogeneity. The isocenter of the magnetic resonance apparatus 10 is also situated there. The Larmor frequency is linked to the strength of the magnetic field by equation (1). In order to excite a specific type of nucleus, depending on the strength of the magnetic field B an RF pulse of a specific frequency f is therefore necessary in order to excite it; when the nucleus returns to its rest position, a magnetic resonance signal is generated as a response signal.

An examination frequency band may be associated with the magnetic field in the isocenter. The examination frequency band includes frequencies that are suitable for exciting protons in the isocenter. To be able to meaningfully use the generated magnetic resonance signals for imaging purposes, they have to be generated in a region of sufficiently high homogeneity, therefore, for example, in the isocenter. Accordingly, response signals, that are generated outside of the isocenter, are not provided for imaging.

As is evident in FIG. 3, the strength of the magnetic field (for example in the y-direction) drifts far outside of the region in the center of the magnet bore 14 that may be used for imaging, so several hundred or thousand ppm deviation from the main magnet field strength may already exist in the vicinity of the inner wall 14. The distribution of the strength of the main magnetic field 13 is customarily symmetrical around the z-direction. “Conventional” imaging is no longer possible in these regions. For example, any local regions of interest in the vicinity of the tunnel wall are usually located (clearly) outside of the homogeneous region of the magnet that may actually be measured.

In an embodiment, the inhomogeneous field in these regions is used and protons (or other nuclei), that are situated in these off-resonant frequency ranges, are purposefully excited. In this way it is possible to advantageously establish whether protons, for example,—i.e. human tissue—are situated in these regions of the magnetic resonance apparatus 10. According to this method, the magnetic resonance apparatus 10 is therefore no longer used (only) as an imaging unit, but as a “sniffer”, in order to ascertain an item of position information as to whether protons, for example, are situated in a specific frequency range of a corresponding spatial region or not.

Advantageously, the method represented in FIG. 2 is therefore suitable for inferring by way of measurement of proton signals that the measuring signal is actually being caused by the patient 15. With comparable optical methods using light barriers it is not possible, for example, to distinguish whether the blocking of a light path was really caused by the patient 15 or other objects, such as coils, pillows, etc.

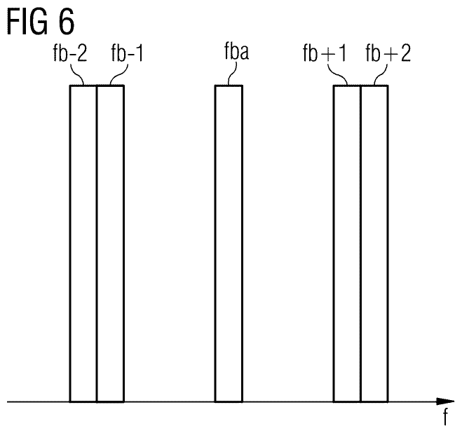

A non-conforming (off-resonant) frequency range with respect to the operating frequency may be excited in S10 by the generated RF transmit pulse. A frequency range may be excited that may be captured with the hardware of the magnetic resonance apparatus, for example with its transmit and/or receive system. This region may have, for example, a deviation with respect to the examination frequency band, for example its center frequency, of ±1,000-2,500 ppm. The regions of ±1,000-2,500 ppm are characterized, by way of example, by 13*+ and the regions of −1,000-2,500 ppm by 13*−. Positive regions 13*+ and negative regions 13*− may either be excited successively or simultaneously by a single RF transmit pulse. FIG. 4 visually represents such frequency bands fb−, fb+ of an RF transmit pulse, that are situated outside of an examination frequency band fba of the magnetic resonance apparatus 10 and/or of the magnetic resonance measurement. The examination frequency band fba includes a center frequency fm here.

Advantageously, the measurement of the imaging magnetic resonance signals during a magnetic resonance measurement of the patient 15 is not disrupted by the measuring in vastly off-resonant regions. Straightforward interleaving of the proposed method for ascertaining the item of position information with simultaneously running imaging magnetic resonance measurements-and thus, for example, sampling in rapid succession—is consequently possible.

The regions 13*− and 13*+ represented in FIG. 3 customarily continue rotationally symmetrically in the z-direction. It is then possible that not only the regions, marked here, in the vicinity of the inner wall W, but also regions more distant from the inner wall, farther out in the z-direction. To prevent response signals from such regions being “confused” with the marked regions and this being inferred, for example a slice-selective excitation is proposed in that a suitable magnetic field gradient is generated in the main magnetic field 13 during the generation of the RF transmit pulse in S10 with the at least one gradient coil. Advantageously, only a specific region is then excited in the z-direction, for example the marked regions 13*− and 13*+ accordingly. This advantageously takes account of the fact that the course of the gradient in these off-center regions is often no longer linear. The excited frequency ranges may be adapted in accordance with these actual courses of the gradient.

Furthermore, an evaluated region is selected via receive antennas, whose position is advantageously known, and the intensity profiles of the received response signal. Since a body coil permanently integrated in the magnetic resonance apparatus 10 usually only includes one or two receive channels, customarily only a relatively rough association is possible when they are used. It is therefore proposed that partial response signals are used that are received by the receive antennas Rx1, Rx2, and Rx3 of the local coil 26.

Advantageously, a resonance is measured directly in S20 after the excitation by the off-resonant RF transmit pulse. If a response signal may be measured and/or the strength of the response signal is above a specified limit value, it is advantageously possible to infer that protons are actually present at locations at which a resonance frequency exists in the magnet bore 14.

S10, S20 and S30 may be repeated for different off-resonant regions. The frequency band of the RF transmit pulses may be varied with repeated generation of the position RF transmit pulse in S10. Advantageously, a plurality of local regions may thus be captured separately in order to ascertain as accurately as possible, using the respective response signals in S30, for example a minimum distance of the patient 15 from the inner wall W as an item of position information.

This is explained in more detail below using FIGS. 5 and 6. Compared to FIG. 3, the spatial regions 13*− are more finely divided in FIG. 5 into the regions 13*−1 and 13*−2, and the regions 13*+ into the regions 13*+1 and 13*+2 respectively. Accordingly, the frequency bands in FIG. 6 of the RF transmit pulses are also divided into fb−2 and fb−2 and fb+2 respectively. A more accurate item of position information may advantageously be ascertained in S30 by way of this division.

Finally, it should again be noted that the methods described in detail above as well as the represented magnetic resonance apparatus are merely exemplary embodiments that may be modified in a wide variety of ways by a person skilled in the art without departing from the scope of the invention. Furthermore, use of the indefinite article “a” or “an” does not preclude the relevant features from also being present multiple times. Similarly, the term “unit” does not preclude the relevant components from being composed of a plurality of cooperating sub-components that may possibly also be spatially distributed.

It is to be understood that the elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present embodiments have been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for ascertaining an item of position information about a position of a patient positioned in a main magnetic field of a magnetic resonance apparatus, wherein the magnetic resonance apparatus comprises at least one transmit antenna, wherein each of the at least one transmit antennas is provided to generate a RF transmit pulse for generating magnetic resonance signals in the patient, wherein the magnetic resonance apparatus comprises at least one receive antenna, wherein the at least one receive antenna is configured to receive a response signal of the RF transmit pulse, the method comprising:
generating an RF transmit pulse with the at least one transmit antenna of the magnetic resonance apparatus, wherein the RF transmit pulse includes a frequency band which is outside of an examination frequency band of the magnetic resonance apparatus;
receiving the response signal of the RF transmit pulse with the magnetic resonance apparatus with the at least one receive antenna of the magnetic resonance apparatus; and
ascertaining the item of position information using the response signal of the RF transmit pulse.

2. The method of claim 1, wherein the item of position information is ascertained during a magnetic resonance measurement of the patient.

3. The method of claim 2, wherein generation of the RF transmit pulse and ascertainment of the item of position information is carried out repeatedly during the magnetic resonance measurement of the patient.

4. The method of claim 3, wherein the frequency band of the RF transmit pulses is varied with repeated generating of the RF transmit pulse and ascertaining of the item of position information.

5. The method of claim 1, wherein the magnetic resonance apparatus includes a magnet bore, inside of which at least one part of the patient is positioned, wherein the item of position information comprises an item of distance information about a distance of the patient from an inner wall of the magnet bore.

6. The method of claim 5, wherein using the item of distance information it is ascertained whether a minimum distance of the patient from the inner wall of the magnet bore is observed.

7. The method of claim 1, wherein the magnetic resonance apparatus comprises at least one gradient coil, wherein a magnetic field gradient is generated in the main magnetic field during generation of the RF transmit pulse with the at least one gradient coil.

8. The method of claim 1, wherein the at least one receive antenna comprises a plurality of receive antennas whose positions relative to the magnetic resonance apparatus are known, wherein each of the plurality of receive antennas is configured to receive a partial response signal of the response signal, wherein the item of position information is ascertained using the partial response signals and the positions of the plurality of receive antennas.

9. The method of claim 8, wherein the plurality of receive antennas are constituent parts of one or more local coils.

10. A magnetic resonance apparatus comprising
at least one transmit antenna, wherein each of the at least one transmit antenna is provided to generate a RF transmit pulse for generating magnetic resonance signals in a patient, wherein the RF transmit pulse includes a frequency band which is outside of an examination frequency band of the magnetic resonance apparatus;
at least one receive antenna, wherein the at least one receive antenna is configured to receive a response signal of the RF transmit pulse; and
a control unit configured to ascertain an item of position information about a position of the patient positioned in a main magnetic field of the magnetic resonance apparatus using the response signal of the RF transmit pulse.

11. The magnetic resonance apparatus of claim 10, wherein the item of position information is ascertained during a magnetic resonance measurement of the patient.

12. The magnetic resonance apparatus of claim 11, wherein generation of the RF transmit pulse and ascertainment of the item of position information is carried out repeatedly during the magnetic resonance measurement of the patient.

13. The magnetic resonance apparatus of claim 12, wherein the frequency band of the RF transmit pulses is varied with repeated generating of the RF transmit pulse and ascertaining of the item of position information.

14. The magnetic resonance apparatus of claim 10, wherein the magnetic resonance apparatus further comprises:
a magnet bore, inside of which at least one part of the patient is positioned, wherein the item of position information comprises an item of distance information about a distance of the patient from an inner wall of the magnet bore.

15. The magnetic resonance apparatus of claim 14, wherein using the item of distance information it is ascertained whether a minimum distance of the patient from the inner wall of the magnet bore is observed.

16. The magnetic resonance apparatus of claim 10, wherein the magnetic resonance apparatus further comprises:
at least one gradient coil, wherein a magnetic field gradient is generated in the main magnetic field during generation of the RF transmit pulse with the at least one gradient coil.

17. The magnetic resonance apparatus of claim 10, wherein the at least one receive antenna comprises a plurality of receive antennas whose positions relative to the magnetic resonance apparatus are known, wherein each of the plurality of receive antennas is configured to receive a partial response signal of the response signal, wherein the item of position information is ascertained using the partial response signals and the positions of the plurality of receive antennas.

18. The magnetic resonance apparatus of claim 17, wherein the plurality of receive antennas are constituent parts of one or more local coils.

19. A non-transitory computer implemented storage medium, including machine-readable instructions stored therein for ascertaining an item of position information about a position of a patient positioned in a main magnetic field of a magnetic resonance apparatus, the machine-readable instructions when executed by at least one processor, cause the processor to:
generate an RF transmit pulse with at least one transmit antenna of the magnetic resonance apparatus, wherein the RF transmit pulse includes a frequency band which is outside of an examination frequency band of the magnetic resonance apparatus;
receive a response signal of the RF transmit pulse with the magnetic resonance apparatus with an at least one receive antenna of the magnetic resonance apparatus; and
ascertain the item of position information using the response signal of the RF transmit pulse.
